(19) United States
(12) Patent Application Publication
(10) Pub. No.: US 2019/0380309 A1
(43) Pub. Date: Dec. 19, 2019

(54) Urine Collection Device

(71) Applicant: Andrea Birchler, East Palestine, OH (US)

(72) Inventor: Andrea Birchler, East Palestine, OH (US)

(21) Appl. No.: 16/009,431

(22) Filed: Jun. 15, 2018

(51) Int. Cl. A01K 23/00; A61D 99/00; A61B 10/00

(52) U.S. Cl. CPC A01K 23/00; A61B 2503/40; A61B 10/007; A61D 99/00

(57) ABSTRACT

A urine collection device for collecting a urine sample from a dog includes a shell, which has a top that is open. A first strap and a second strap are coupled to and extend from the shell proximate to the top. A first coupler is coupled to the first strap distal from the shell. A second coupler is coupled to the second strap distal from the shell. The second coupler is complementary to the first coupler. The first strap and the second strap are configured to position around an abdomen of a dog, positioning the second coupler to couple to the first coupler with the top of the shell positioned over a urethral meatus of the dog. The shell is configured to collect urine from the dog.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to collection devices and more particularly pertains to a new collection device for collecting a urine sample from a dog.

### BRIEF SUMMARY OF THE INVENTION

An embodiment of the disclosure meets the needs presented above by generally comprising a shell, which has a top that is open. A first strap and a second strap are coupled to and extend from the shell proximate to the top. A first coupler is coupled to the first strap distal from the shell. A second coupler is coupled to the second strap distal from the shell. The second coupler is complementary to the first coupler. The first strap and the second strap are configured to position around an abdomen of a dog, positioning the second coupler to couple to the first coupler with the top of the shell positioned over a urethral meatus of the dog. The shell is configured to collect urine from the dog.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a urine collection device according to an embodiment of the disclosure.

FIG. 2 is a bottom view of an embodiment of the disclosure.

FIG. 3 is a top view of an embodiment of the disclosure.

FIG. 4 is a side view of an embodiment of the disclosure.

FIG. 5 is a detail view of an embodiment of the disclosure.

FIG. 6 is an in-use view of an embodiment of the disclosure.

### DETAILED DESCRIPTION OF THE INVENTION

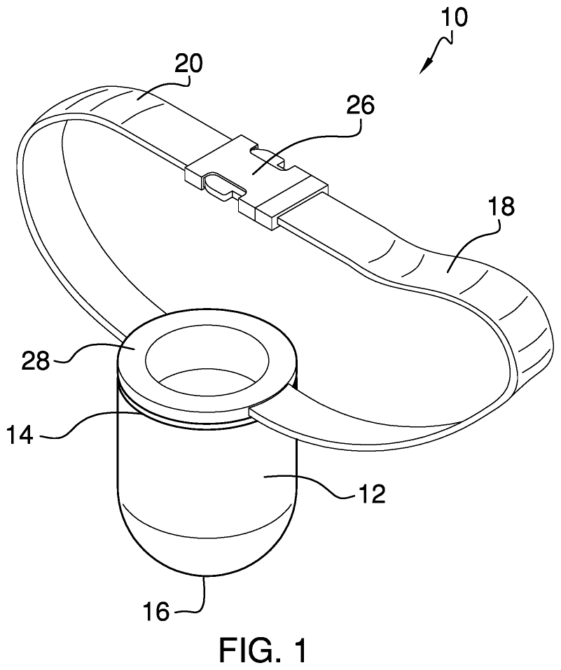

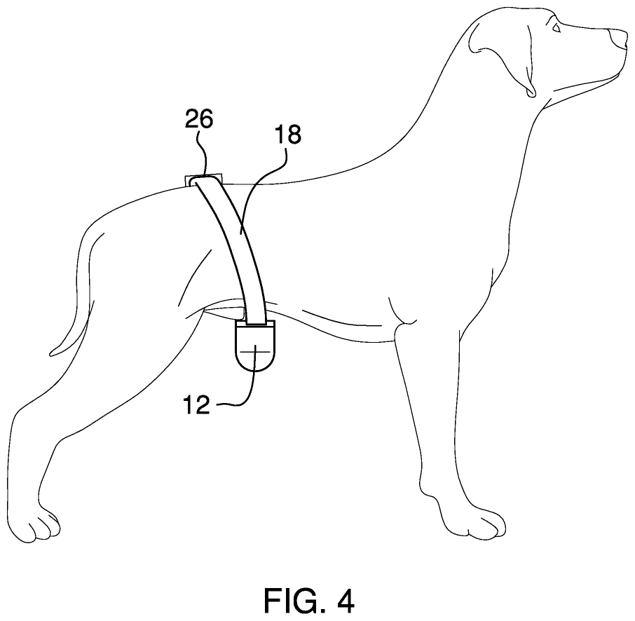

With reference now to the drawings, and in particular to FIGS. 1 through 6 thereof, a new collection device embodying the principles and concepts of an embodiment of the disclosure and generally designated by the reference numeral 10 will be described.

As best illustrated in FIGS. 1 through 6, the urine collection device 10 generally comprises a shell 12, which has a top 14 that is open. The shell 12 has a bottom 16 that is arcuate. As will become apparent, the bottom 16 being rounded minimizes discomfort to a dog to which the shell 12 is coupled.

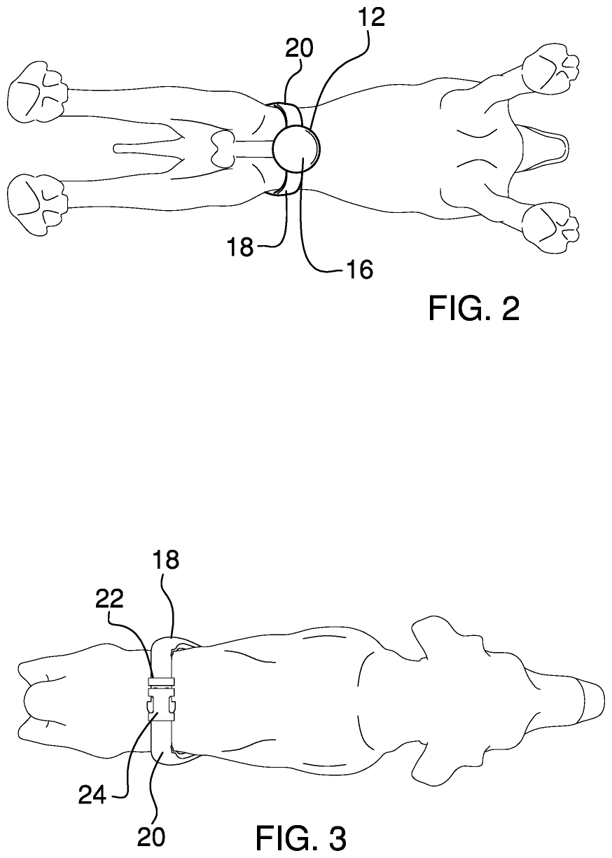

A first strap 18 and a second strap 20 are coupled to and extend from the shell 12 proximate to the top 14. A first coupler 22 is coupled to the first strap 18 distal from the shell 12. A second coupler 24 is coupled to the second strap 20 distal from the shell 12. The second coupler 24 is complementary to the first coupler 22. The first strap 18 and the second strap 20 are configured to position around an abdomen of a dog, as shown in FIGS. 2, 3 and 4. The second coupler 24 is positioned to couple to the first coupler 22 with the top 14 of the shell 12 positioned over a urethral meatus of the dog. The shell 12 is configured to collect urine from the dog.

Urine samples are often required for health screening and to make informed medical decisions in the care of the dogs. The device 10 provides a nonintrusive means to collect the urine sample and entails minimal discomfort to the dog.

The first coupler 22 and the second coupler 24 comprise a side release buckle 26. The first coupler 22 is slidably coupled to the first strap 18 so that the first coupler 22 is selectively positionable on the first strap 18 relative to the shell 12. This configuration allows the device 10 to be sized to fit dogs of a variety of sizes.

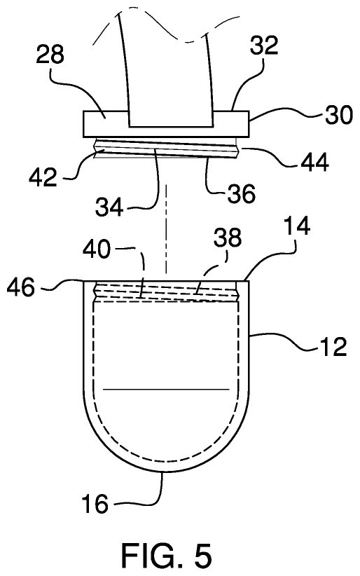

The device 10 comprises a ring 28. The first strap 18 and the second strap 20 are coupled to an outer perimeter 30 of the ring 28 proximate to an upper end 32 of the ring 28, as shown in FIG. 1. A first connector 34 is coupled to the ring 28 proximate to a lower end 36 of the ring 28, as shown in FIG. 5. A second connector 38 is coupled to the shell 12 proximate to the top 14. The second connector 38 is complementary to the first connector 34. The second connector 38 is positioned to selectively couple to the first connector 34 to removably couple the shell 12 to the ring 28.

The ability to removably couple the shell 12 to the ring 28 allows a user to remove the shell 12 from the dog while the ring 28, the first strap 18, and the second strap 20 remain positioned on the dog. The removable nature of the shell 12 also permits sterilization of the shell 12, or substitution of a new sterile shell 12, prior to again coupling the shell 12 to the ring 28. This configuration also permits use of the device 10 sequentially with an unlimited number of dogs.

The second connector 38 comprises internal threads 40 positioned in the shell 12 proximate to the top 14, as shown in FIG. 5. The first connector 34 comprises external threads 42 positioned on the ring 28 proximate to the lower end 36 of the ring 28. The shell 12 is positioned to removably threadedly couple to the ring 28.

A recess 44 extends into the ring 28 from the lower end 36 and the outer perimeter 30. The external threads 42 are positioned in the recess 44. The top 14 of the shell 12 has an outer circumference 46. The outer circumference 46 is substantially complementary to the outer perimeter 30 of the ring 28, again to minimize discomfort to the dog.

The embodiment detailed above is most appropriate for use with male dogs. An alternative embodiment, described below, is better suited for use with female dogs.

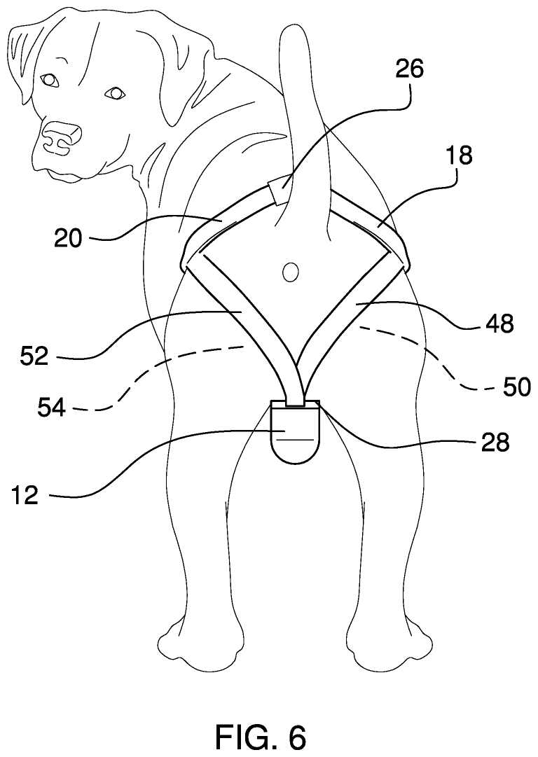

In this alternative embodiment of the invention, a third strap 48 is coupled to and extends between the first strap 18 and the ring 28, as shown in FIG. 6. The third strap 48 is coupled to the first strap 18 between the first coupler 22 and the ring 28 to define a first loop 50.

A fourth strap 52 is coupled to and extends between the second strap 20 and the ring 28. The fourth strap 52 is coupled to the second strap 20 between the second coupler 24 and the ring 28 define a second loop 54. The first loop 50 and the second loop 54 are configured to insert a right rear leg and a left rear leg of a female dog, respectively. The first strap 18 and the second strap 20 are configured to position around the abdomen of the female dog. The second coupler 24 is positioned to couple to the first coupler 22 with the top 14 of the shell 12 positioned over the urethral meatus of the female dog. The shell 12 is configured to collect the urine from the female dog.

In use, the first strap 18 and the second strap 20 are positioned around the abdomen of the dog, positioning the second coupler 24 to couple to the first coupler 22 with the top 14 of the shell 12 positioned over the urethral meatus of the dog. The user then waits for the dog to urinate. The shell 12 is configured to collect the urine from the dog.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A urine collection device comprising:
a shell having a top, the top being open;
a first strap coupled to and extending from the shell proximate to the top;
a second strap coupled to and extending from the shell proximate to the top;
a first coupler coupled to the first strap distal from the shell; and
a second coupler coupled to the second strap distal from the shell, the second coupler being complementary to the first coupler wherein the first strap and the second strap are configured for positioning around an abdomen of a dog positioning the second coupler for coupling to the first coupler with the top of the shell positioned over a urethral meatus of the dog wherein the shell is configured for collecting urine from the dog.

2. The device of claim 1, further including the shell having a bottom, the bottom being arcuate.

3. The device of claim 1, further including the first coupler and the second coupler comprising a side release buckle.

4. The device of claim 1, further including the first coupler being slidably coupled to the first strap such that the first coupler is selectively positionable on the first strap relative to the shell.

5. The device of claim 1, further comprising:
a ring, the first strap and the second strap being coupled to an outer perimeter of the ring proximate to an upper end of the ring;
a first connector coupled to the ring proximate to a lower end of the ring; and
a second connector coupled to the shell proximate to the top, the second connector being complementary to the first connector wherein the second connector is positioned for selectively coupling to the first connector for removably coupling the shell to the ring.

6. The device of claim 5, further including the second connector comprising internal threads positioned in the shell proximate to the top, the first connector comprising external threads positioned on the ring proximate to the lower end of the ring wherein the shell is positioned for removably threadedly coupling to the ring.

7. The device of claim 6, further including a recess extending into the ring from the lower end and the outer perimeter, the external threads being positioned in the recess, the top of the shell having an outer circumference, the outer circumference being substantially complementary to the outer perimeter of the ring.

8. The device of claim 5, further including comprising:
a third strap coupled to and extending between the first strap and the ring, the third strap being coupled to the first strap between the first coupler and the ring defining a first loop; and
a fourth strap coupled to and extending between the second strap and the ring, the fourth strap being coupled to the second strap between the second coupler and the ring defining a second loop wherein the first loop and the second loop are configured for inserting a right rear leg and a left rear leg of a female dog, respectively, wherein the first strap and the second strap are configured for positioning around the abdomen of the female dog positioning the second coupler for coupling to the first coupler with the top of the shell positioned over the urethral meatus of the female dog wherein the shell is configured for collecting the urine from the female dog.

9. A urine collection device comprising:
a shell having a top, the top being open, the shell having a bottom, the bottom being arcuate;
a first strap coupled to and extending from the shell proximate to the top;
a second strap coupled to and extending from the shell proximate to the top;
a first coupler coupled to the first strap distal from the shell;
a second coupler coupled to the second strap distal from the shell, the second coupler being complementary to the first coupler wherein the first strap and the second strap are configured for positioning around an abdomen of a dog positioning the second coupler for coupling to the first coupler with the top of the shell positioned over a urethral meatus of the dog wherein the shell is configured for collecting urine from the dog, the first coupler and the second coupler comprising a side release buckle, the first coupler being slidably coupled to the first strap such that the first coupler is selectively positionable on the first strap relative to the shell;
a ring, the first strap and the second strap being coupled to an outer perimeter of the ring proximate to an upper end of the ring;
a first connector coupled to the ring proximate to a lower end of the ring;
a second connector coupled to the shell proximate to the top, the second connector being complementary to the first connector wherein the second connector is positioned for selectively coupling to the first connector for removably coupling the shell to the ring, the second connector comprising internal threads positioned in the shell proximate to the top, the first connector comprising external threads positioned on the ring proximate to the lower end of the ring wherein the shell is positioned for removably threadedly coupling to the ring; and
a recess extending into the ring from the lower end and the outer perimeter, the external threads being positioned in the recess, the top of the shell having an outer circumference, the outer circumference being substantially complementary to the outer perimeter of the ring.

10. The device of claim 9, further comprising:
a third strap coupled to and extending between the first strap and the ring, the third strap being coupled to the first strap between the first coupler and the ring defining a first loop; and
a fourth strap coupled to and extending between the second strap and the ring, the fourth strap being coupled to the second strap between the second coupler and the ring defining a second loop wherein the first loop and the second loop are configured for inserting a right rear leg and a left rear leg of a female dog, respectively, wherein the first strap and the second strap are configured for positioning around the abdomen of the female dog positioning the second coupler for coupling to the first coupler with the top of the shell positioned over the urethral meatus of the female dog wherein the shell is configured for collecting the urine from the female dog.
